(19) United States
(12) Patent Application Publication
(10) Pub. No.: US 2024/0399379 A1
(43) Pub. Date: Dec. 5, 2024

(54) METHOD TO CONDUCT POLYMERASE CHAIN REACTION AMPLIFICATION OF BIOLOGICAL SAMPLE WITHOUT NUCLEIC ACIDS ISOLATION

(71) Applicant: UNIVERSITY OF CENTRAL FLORIDA RESEARCH FOUNDATION, INC., Orlando, FL (US)

(72) Inventors: Kiminobu SUGAYA, Orlando, FL (US); Vaidya MANJUSHA, Orlando, FL (US)

(21) Appl. No.: 18/697,604

(22) PCT Filed: Sep. 28, 2022
(86) PCT No.: PCT/US2022/045036

§ 371 Date: Apr. 1, 2024

Related U.S. Application Data
(60) Provisional application No. 63/304,970, filed on Jan. 31, 2022.
(60) Provisional application No. 63/250,412, filed on Sep. 30, 2021.

(51) Int. Cl. B01L 7/00; C12Q 1/6844

(52) U.S. Cl. CPC B01L 7/52; C12Q 1/6844; B01L 2300/023; B01L 2300/025; B01L 2300/0663; B01L 2300/0681; B01L 2300/126; B01L 2300/1822; B01L 2300/1894

(57) ABSTRACT

Disclosed is a method to amplified nucleic acids by polymerase chain reaction (PCR) without isolation or purification from the biological samples. All the reactions associated with reverse transcriptase qPCR or real-time qPCR are completed on a filter paper chamber using a handheld Real-time PCR device. The process requires a very small amount of sample since there is no loss of materials associated with isolation and purification steps. The amplification process is faster than a normal tube-based PCR reaction because the thin chamber makes heating and cooling process fast. This technique is applicable in the rapid detection of pathogenic viruses from body fluids, such as COVID-19 detection in saliva. The technology is valuable for quantitative detection of nucleic acids that are established markers of diseases. Novel use of a piece of filter paper to perform sample collection, storage, and conduct direct PCR amplification without any extraction of the sample will allow us to test infections and the diseases anywhere by anybody without the requirement of technical knowledge or skills.

### BACKGROUND

Amplification of nucleic acids via polymerase chain reaction (PCR) is a well-established technique used for diagnostic, prognostic and predictive purposes of a disease. Various types of PCR amplification methods such as reverse transcriptase qPCR and real-time qPCR are employed to accomplish the goal. Typically, DNA or RNA, the template material to be amplified, is acquired by lengthy extraction and purification procedures from the body fluids. The process needs technical knowledge and skills for accuracy of results. Embodiments in this disclosure bypasses this tedious and time-consuming isolation process. All the steps such as sample collection, nucleic acid extraction, purification, storage, and PCR amplification are performed on a piece of filter paper. The specimens are directly applied to the filter paper and the same piece of paper is used in PCR reaction. Because there is no loss of sample during the process, small amounts as low as 5 μL are enough for amplification. If a special flat chamber used to accommodate the filter paper, thermocycling amplification periods can also be cut down compared to the usual time required for amplification. The technique of PCR amplification on a paper is useful in identification of unique and pathogenic DNA/RNA sequences to diagnose a disease. The technique is also useful for qualitative and quantitative analysis of marker DNA/RNA sequences in diseases like cancers for diagnostic, prognostic and predictive purposes.

Analysis of nucleic acids in body fluids using this method can reduce the need for severely invasive procedures. Most importantly, once streamlined, the entire process from collection of the samples to analysis and inference of the results can be performed by an individual without prerequisite of technical knowledge and specific skills.

Disclosed is a handheld machine to accept this special type of flat chamber. With this machine anybody can do PCR anywhere without any special equipment.

### BRIEF DESCRIPTION OF THE DRAWINGS

The present embodiments are illustrated by way of example, and not by way of limitation, in the figures of the accompanying drawings.

The following figures are illustrative only, and are not intended to be limiting

FIG. 1 show the gel electrophoresis of a standard PCR. Standard PCR amplification of DNA from conditioned media on filter paper without nucleic acid isolation. FIG. 1A shows gel electrophoresis of the PCR product along with the filter paper discs used for amplification. Due to the retention of PCR product, the filter paper shows fluorescence. Some of the PCR products were migrate out to the gel. FIG. 1B shows filter paper discs without template or PCR amplification do not show any fluorescent signals.

FIG. 2 is the thermocycling run method for qPCR.

FIG. 3 shows the amplification plots (A) and CT mean values of the samples (B) (FP—filter paper, q-qPCR).

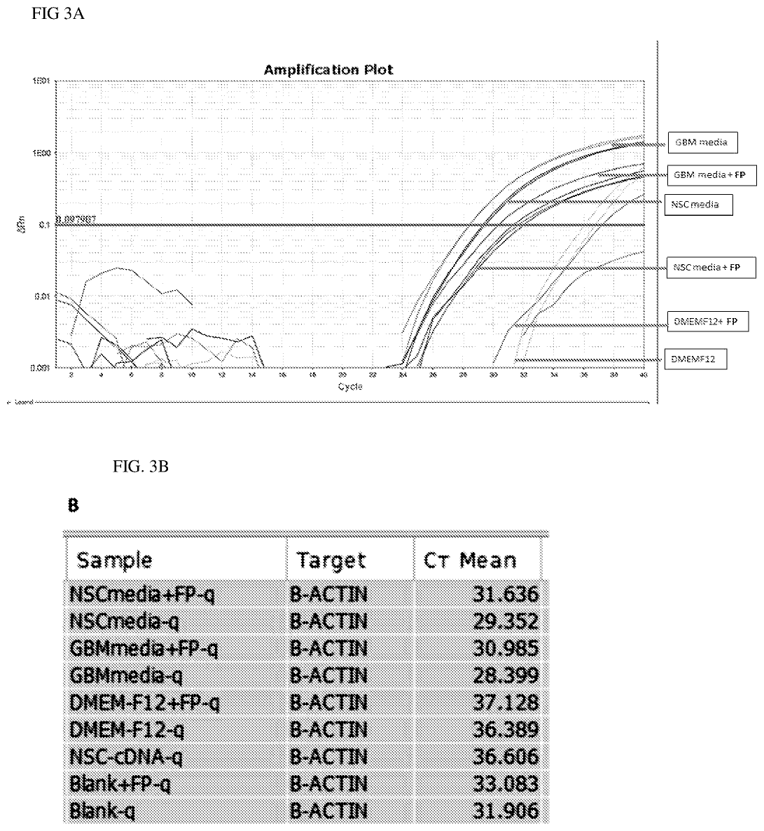

FIG. 4 is a graph based on the RFU values of the samples at excitation and emission wavelengths of 475/530 nm.

FIG. 5 is the run method for isothermal-PCR.

FIG. 6 are the amplification and melt curve plots of HCoV229E cDNA. The amplification curve and the Ct value of 6.867 signifies the isothermally amplified templet on filter paper. The sample without filter paper showed no amplification.

FIG. 7 are the amplification and melt curve plots of HCoV229E cDNA with a direct use of viral media. The amplification curve and the Ct value of 23.291 signifies the isothermally amplified templet on filter paper. The sample without filter paper showed no amplification.

FIG. 8 shows the filter paper for paper-based PCR.

FIG. 9 shows the structure of the handheld Real-time PCR device.

FIG. 10 shows the structure of the PCR chamber.

FIG. 11 are pictures of the handheld Real-time PCR device.

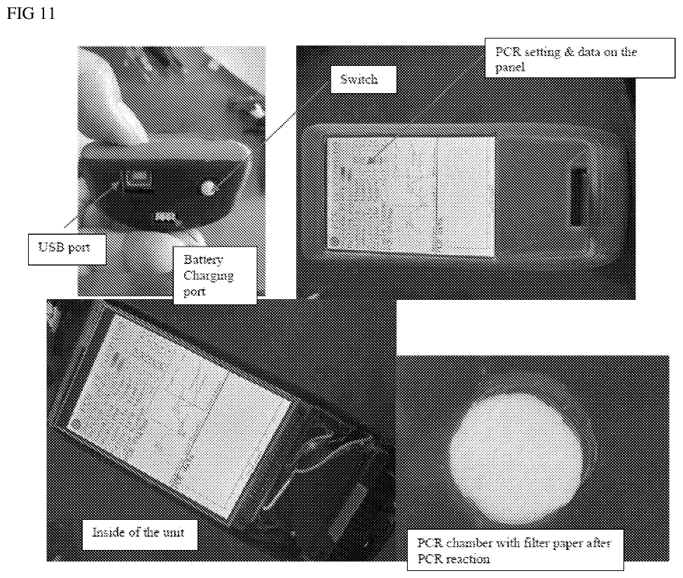

FIG. 12 is a picture of the handheld Real-time PCR device displaying the run method and data from a SYBR green real time PCR.

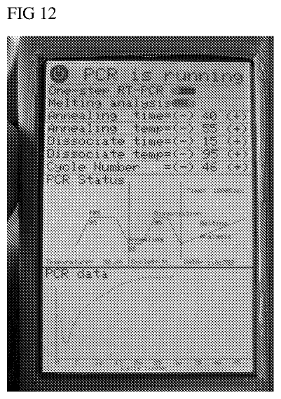

### DEFINITIONS

Unless defined otherwise, all technical and scientific terms used herein have the same meaning as commonly understood by one of ordinary skill in the art to which this invention belongs. Although any methods and materials similar or equivalent to those described herein can be used in the practice or testing of the invention, the preferred methods and materials are now described. All publications mentioned herein are incorporated herein by reference.

Generally, nomenclatures used in connection with, and techniques of, cell and tissue culture, molecular biology, immunology, microbiology, genetics, protein, and nucleic acid chemistry and hybridization described herein are those well-known and commonly used in the art. The methods and techniques of the present invention are generally performed according to conventional methods well known in the art and as described in various general and more specific references that are cited and discussed through the present specification unless otherwise indicated.

The term “amplifying” or “amplification” a nucleic acid sequence generally refers to the production of a plurality of nucleic acid copy molecules having that sequence from a target nucleic acid wherein primers hybridize to specific sites on the target nucleic acid molecules in order to provide an initiation site for extension by a polymerase, e.g., a DNA polymerase. Amplification can be carried out by any method generally known in the art, such as but not limited to: standard PCR, real-time PCR, long PCR, hot start PCR, qPCR, Reverse Transcription PCR and Isothermal Amplification.

The term “amplification cycles” refers to the series of temperature and time adjustments in PCR. Each amplification cycle includes steps for template denaturation, primer annealing and primer extension. Each amplification cycle theoretically doubles the amount of targeted sequence in the reaction.

The term “detecting” or “detection” as used herein relates to a test aimed at assessing the presence or absence of a target nucleic acid in a sample.

The term “filter” or “filter paper” refers to a semi-permeable paper barrier placed perpendicular to a liquid or air flow. Filter papers are widely used in the arts across many different fields, from biology to chemistry.

The terms “labeling agent” or “label” refers to groups that make a nucleic acid, in particular oligonucleotides or modified oligonucleotides, as well as any nucleic acids bound thereto, distinguishable from the remainder of the sample. Useful labels in the context of the invention are e.g. fluorescent labels, which may be fluorescent dyes such as for instance a fluorescein dye, a rhodamine dye, a cyanine dye, or a coumarin dye. Useful fluorescent dyes in the context of the invention are e.g. FAM, HEX, JA270, CAL635, Coumarin343, Quasar705, Cyan500, CY5.5, LC-Red 640, LC-Red 705, TAMRA, SYBR, EvaGreen or CY5. However, any label which can render the amplified target sequence nucleic acid be detected can be used for methods according to this invention.

The terms “nucleic acid,” “polynucleotide,” and “oligonucleotide” are used interchangeably and refer to a deoxyribonucleotide or ribonucleotide polymer, in linear or circular conformation, and in either single- or double-stranded form. For the purposes of the present disclosure, these terms are not to be construed as limiting with respect to the length of a polymer. The terms can encompass known analogues of natural nucleotides, as well as nucleotides that are modified in the base, sugar and/or phosphate moieties (e.g., phosphorothioate backbones). In general, an analogue of a particular nucleotide has the same base-pairing specificity; i.e., an analogue of A will base-pair with T.

A “target nucleic acid” or “template nucleotide” is a polymeric compound of nucleotides as known to the expert skilled in the art. “Target nucleic acid” is used herein to denote a nucleic acid in a sample which should be analyzed, i.e. the presence, non-presence and/or amount thereof in a sample should be determined. The target nucleic acid may be a genomic sequence, e.g. part of a specific gene, or RNA. In other embodiments, the target nucleic acid may be viral or bacterial. Target nucleic acids can comprise subgroups with distinct sequence variations or distinct individual mutations in the amplicon region. This is especially the case for nucleic acids of pathogens like viruses which show significant genetic variation due to high mutation or recombination rates and lacking repair mechanisms.

The term “polymerase chain reaction” or “PCR” refers to a technique widely used in molecular biology to amplify a nucleic acid by in vitro enzymatic replication. PCR can refer to such methods generally known in the art, such as but not limited to: standard PCR, real-time PCR, long PCR, hot start PCR, qPCR, Reverse Transcription PCR and Isothermal Amplification.

The term “PCR cocktail” as used herein means a solution for PCR including: deoxynucleotide triphosphates (dNTPs); a buffer solution providing a suitable chemical environment for optimum activity and stability of the DNA polymerase and/or reverse transcriptase; divalent cations, typically magnesium ions (Mg2+); and monovalent cation potassium ions.

The term “primer” is used herein as known to the expert skilled in the art and refers to oligomeric compounds, primarily to oligonucleotides, but also to modified oligonucleotides, that are able to prime DNA synthesis by a template-dependent DNA polymerase, i.e. the 3′-end of the primer provides a free 3′-OH group where further nucleotides may be attached by a template-dependent DNA polymerase establishing 3′- to 5′-phosphodiester linkage whereby deoxynucleoside triphosphates are used and whereby pyrophosphate is released.

The terms “quantifying” and “quantification” as used herein relates to a test aimed at assessing the amount of a target nucleic acid in a sample

The term “reaction buffer” refers to a buffer that provides a suitable chemical environment for activity of DNA polymerase. The buffer pH is usually between 8.0 and 9.5 and is often stabilized by Tris-HCl. A common component in reaction buffers is potassium ion (K+) from KCl, which promotes primer annealing. At times, ammonium sulfate (NH4) 2SO4 may replace KCl in the buffer. The ammonium ion (NH4+) has a destabilizing effect, especially on weak hydrogen bonds between mismatched primer-template base-pairing, thereby enhancing specificity. DNA polymerases often come with reaction buffers that have been optimized for robust enzyme activity; therefore, it is recommended to use a provided buffer to achieve optimal PCR results.

A “sample” is any material that can be subjected to a diagnostic assay and generally refers to the medium possibly containing the target nucleic acid. The “sample” is in some embodiments derived from a biological source. The sample can be e.g. a clinical sample. In some embodiments, said sample is derived from a human and is a body liquid or biopsy sample. In some embodiments of the invention, the sample is human whole blood or serum, blood plasma, urine, sputum, sweat, breast milk, semen, intraocular fluid, genital or buccal or nasal swabs, pipettable stool, solubilized tissue samples, or spinal fluid or the like. A sample can be pipetted or converted to a pipettable form, such that the term “sample” comprises homogeneous or homogenized liquids, but also emulsions, suspensions and the like. A sample may also e.g. be an originally solid sample (i.e. tissue sample) which is subjected to a solubilization treatment for extraction and purification of nucleic acids.

The term “thermostable and strand-displacement DNA polymerase” as used herein is an enzyme capable of synthesizing nucleic acids from smaller elements such as nucleotides. As used herein a “stand-displacement” enzyme has the ability to displace downstream DNA encountered during synthesis. As used herein an “thermostable” enzyme is stable to heat, is heat resistant, and retains sufficient activity to effect subsequent polynucleotide extension reactions and does not become irreversibly denatured (inactivated) when subjected to the elevated temperatures for the time necessary to effect denaturation of double-stranded nucleic acids. The heating conditions necessary for nucleic acid denaturation are well known in the art and are exemplified in, e.g., U.S. Pat. Nos. 4,683,202, 4,683,195, and 4,965,188. As used herein, a thermostable polymerase is suitable for use in a temperature cycling reaction such as the polymerase chain reaction (“PCR”) and such polymerases are known to those of skill in the art.

### DETAILED DESCRIPTION

The present disclosure is based on the finding that by employing various PCR amplification methods, nucleic acids were successfully amplified on a piece of filter paper containing a drop of sample with COVID virus (HCoV-229E) as a template. A viral sample was applied directly to the paper disk and heated it up to 95° C. for 5 min to destruct the virus. This method is applicable to any biological vessel containing nucleotides. An advantage of embodiments described herein, in the case of harmful viruses, after their collection and storage, the inactivation/destruction of viral particles can be carried out on the same filter paper without the need for frequent handling of the viral sample. This eliminates any chance of the researcher getting exposed to dangerous virus. Additionally, with certain method embodiments, the thermo-cycling time can be reduced significantly, thereby delivering quicker and accurate results. In addition to amplifying RNA of COVID virus, the DNA from as low as 5 ul of conditioned media of Glioblastoma multiforme (GBM) cells and human neural stem cells (NSC) was successfully amplified on filter paper using standard and qPCR techniques, making DNA isolation and purification obsolete. Employing this technique and disease-specific primers for marker genes, qualitative and quantitative analysis of marker DNA/RNA sequences of exosomes in the patient's body fluids can be performed for diagnostic, prognostic and predictive purposes of various diseases including cancer. The body fluids can be acquired with completely non-invasive (e.g. saliva, urine) or minimally invasive (e.g. blood) ways thereby avoiding painfully invasive procedures.

According to one embodiment, provided is a method for amplifying and detecting one or more nucleic acid targets in a sample that involves collecting a sample, applying the sample to a filter paper, heat inactivating the sample on the filter paper, applying a PCR cocktail to the sample on the filter paper, inserting the filter paper into a handheld Real-time PCR device, performing one or more amplification cycles using the handheld Real-time PCR device, and detecting the presence or absence of one or more nucleic acid target.

In another embodiment, provided is a method for amplifying and quantifying one or more nucleic acid targets in a sample comprising collecting a sample, applying the sample to a filter paper, heat inactivating the sample, applying a PCR cocktail and a labeling agent to the sample on the filter paper, inserting the filter paper into a handheld Real-time PCR device, performing one or more amplification cycles using the handheld Real-time PCR device to form labeled amplification products for each of the one or more nucleic acid targets, and detecting and quantifying a signal from the labeled amplification products.

In yet another embodiment, disclosed is a method for amplifying and detecting one or more nucleic acid targets in a sample comprising: collecting a sample on a filter paper comprising one or more capture molecules immobilized thereon, heat inactivating the sample on the filter paper, applying a PCR cocktail to the sample on the filter paper, inserting the filter paper into a Real-time PCR device, performing one or more amplification cycles using the Real-time PCR device, and detecting the presence or absence of one or more nucleic acid target.

Another method embodiment pertains to a method for amplifying and quantifying one or more nucleic acid targets in a sample comprising collecting a sample on a filter paper comprising one or more capture molecules immobilized on a surface thereof, heat inactivating the sample, applying a PCR cocktail and a labeling agent to the sample on the filter paper, inserting the filter paper into a Real-time PCR device, performing one or more amplification cycles using the Real-time PCR device to form labeled amplification products for each of the one or more nucleic acid targets, and detecting and quantifying a signal from the labeled amplification products.

In specific embodiments, the capture molecule is an antibody and/or aptamer, that optionally binds to an antigen such as a nucleic acid or amino acid sequence. The antigen may also, but not necessarily, form a microbe such as virus, fungi or bacterium, wherein optionally, the microbe is a pathogen (e.g., SARs-COV-2). In a specific embodiment, the pathogen is a virus comprising a surface protein (e.g. spike protein of SARS-COV-2).

Examples of samples for amplification include but art not limited to blood, serum, saliva, sputum, mucus, urine, milk, semen, vaginal fluid, or any liquid containing nucleic acids, or any sample collected from a surface, such as by swipe sampling. An example of a PCR cocktail is one that includes at least one primer pair for priming amplification of a nucleic acid target, an enzyme for amplifying the nucleic acid targets, dNTPs, and a reaction buffer. In a specific embodiment, inactivating the sample involves incubating the sample on the filter paper at 95° C. for at least 5 minutes.

In some embodiments, the enzyme for amplifying the nucleic acid targets comprises a heat resistant and strand-displacement DNA polymerase. Labeling agents can be those commercially available, and in a specific embodiment, the labeling agent used is afluorescent label selected from the group comprising SYBR™ Green or EvaGreen®. In a specific embodiment, the amplification cycles do not exceed a temperature of 70° C.

According to another embodiment, provided is a handheld Real-time PCR device, comprising:
a. a PCR chamber to hold a filter containing a sample and a PCR cocktail: comprising a peltier device connected to a heatsink and a cooling fan;
b. an optical detection system comprising a blue LED, a photosensor, an excitation filter, and an emission filter;
c. a control system comprising a Peltier and cooling fan controller, a LED and photosensor control board, a master control board connecting to an exterior touch control panel, and a communication interface to interact with other devices; and
d. a power system comprising a power source, an external charging port, and a power control switch.
   The PCR chamber may include an opening allowing for inserting the filter containing a sample and a PCR cocktail. The heatsink and cooling fan may be configured to direct heat from the PCR chamber to outside the device. The optical detection system may be configured to excite and detect a fluorescence signal from a sample within the PCR chamber.

In a specific embodiments, one or more of the following options are provided: the excitation filter comprises a band pass 480 nm filter, the emission filter may comprise a band pass 520 nm filter, the exterior touch control panel displays PCR settings and data, or is associated with a separate device that communicates with the master control board, wherein the separate device is optionally a smartphone, the power source comprises a rechargeable battery or non-rechargeable battery, wherein the battery last for at least 2, at least 3, at least 4, at least 5 or at least 6 PCR reactions, wherein the communication interface is selected from the group comprising serial, USB, Bluetooth, wifi, cellular, satellite, or combinations thereof.

Another embodiment pertains to a cellulose filter paper comprising an antibody and/or aptamer immobilized onto a surface thereof, wherein the antibody and/or aptamer is specific to a microbe protein (e.g. SARS-COV-2).

### Filter Paper

In some embodiments, disclosed is a method to conduct PCR amplification of biological samples on filter paper without nucleic acid isolation. The method does not require isolating nucleic acids or removing cell debris or macromolecules by centrifugation. Each stage of PCR is performed on the filter paper including sample collection, nucleic acid extraction, purification, storage, and PCR amplification. The sample is not transferred to multiple containers, and the same filter paper is used throughout. This prevents loss of sample and allows for amplifying amounts as low as 5 μL of sample. This method is applicable to any biological vessel containing nucleotides. In some embodiments, the sample contains harmful viruses. After their collection and storage, the inactivation and destruction of viral particles can be carried out on the same filter paper without the need for frequent handling of the viral sample. This eliminates any chance of the researcher getting exposed to dangerous virus.

In preferred embodiments, the filter paper is quantitative filter paper. Quantitative filter paper is made from high-quality alpha cotton cellulose, washed in hydrochloric acid and neutralized in an ultrapure water rinse. Quantitative filter paper leaves no more than 0.01% ash when burned. The quantitative filter paper can retain particles 5 to 10 μm in diameter and has a medium porosity. The quantitative filter paper allows for the immobilization of a sample thereby concentrating it in a confined space to increase the sensitivity. The filter paper holds molecules in the sample inside by non-specific binding and prevents the template nucleotides and PCR reagents from being exposed to the molecules inhibiting PCR reaction. While the PCR nucleotide template molecules are exposed only to the PCR reagents. Due to this feature, the addition of the template sample followed by the PCR cocktail to the filter paper assures an effective PCR reaction and results in a successful and sensitive amplification of the target nucleic acid without isolation and purification. Use of thin filter paper as a reaction chamber makes PCR reaction much faster than regular tube-based PCR because the easiness of heating and cooling cycle.

In certain embodiments, the color of the filter paper is selected to optimize visualization of a signal such as fluorescent signals. In one example, the filter paper is white in color. In other embodiments, the filter paper is other than white. In a specific embodiment, the filter paper is of a dark color, such as black, brown, blue or grey or some combination thereof. The grey may comprise a hex triplet including but not limited to: #DCDCDC. #D3D3D3, #C0C0CO, #A9A9A9, #808080, #696969, #778899, #708090, or #2F4F4F. The color of the filter paper may be one selected to optimize a contrast with the dye or fluorescent signal used.

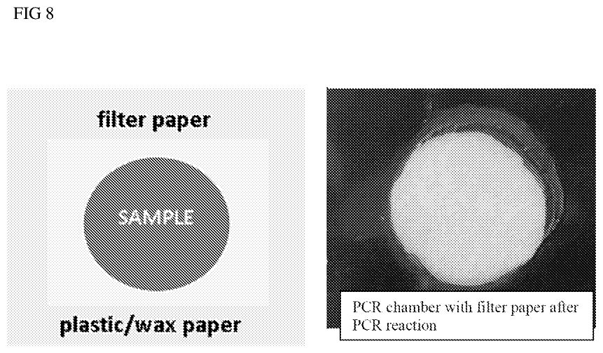

The present disclosure also includes various PCR amplification methods using the filter paper. The nucleic acids are amplified on a piece of filter paper containing a drop of sample with COVID virus (HCoV-229E) as a template. Viral samples are applied directly to the paper disk and heated it up to 95° C. for 5 min to destruct the virus. Additionally, with the method, the thermo-cycling time can be reduced significantly, thereby delivering quicker and accurate results. A representation of the sample on the filter paper is shown in FIG. 8 along with the filter paper under UV light post PCR. As noted above, RNA of COVID virus, and DNA from as low as 5 μl of conditioned media of Glioblastoma multiforme (GBM) cells and human neural stem cells (NSC) was successfully amplified on filter paper using standard and qPCR techniques, making DNA isolation and purification obsolete. Employing disclosed method embodiments and disease-specific primers for marker genes, qualitative and quantitative analysis of marker DNA/RNA sequences of exosomes in the patient's body fluids can be performed for diagnostic, prognostic and predictive purposes of various diseases including cancer. The body fluids can be acquired with completely non-invasive (e.g. saliva, urine) or minimally invasive (e.g. blood) ways thereby avoiding painfully invasive procedures.

### Detailed Description of Handheld Real-time PCR device

The present disclosure also includes self-contained nucleotide-based bioanalytical systems incorporated into portable devices that incorporate the filter for nucleotide capture, detection, and quantitation using the methods of the present disclosure. Advances in system miniaturization make it possible to combine sample preparation, amplification and detection in a single portable device that may include chambers, channels, and/or heating or cooling elements to store and manipulate samples and reagents and/or scan particles as described for various embodiments of the multiplexed end point quantitative PCR assays. The Real-time PCR device contains a special flat chamber to accommodate PCR performed on a filter paper.

Hand-held or portable devices can be used in point-of-care facilities such as doctor's offices or hospitals, veterinarian's offices, pharmacies, diagnostics labs, and clinics; and also for detecting biological threats in civilian or military areas. A review of portable nucleic acid bioanalytical systems is provided by T. M. Lee et al., DNA-based bioanalytical microsystems for handheld device applications, Analytica Chemica, Acta 556 (2006) 26-37; see also T. Ray, UK Startup DNA Electronics Developing Handheld Device to Detect Genetic Risk for Drug AEs, Pharmacogenomics Reporter—Feb. 25, 2009; and K. P. O′Connell et al., Testing of the Bio-Seeq (Smiths Detection Handheld PCR Instrument): Sensitivity, Specificity, and Effect of Interferents on Bacillus Assay Performance, Edgewood Chemical Biological Center Aberdeen Proving Ground, MD Report No. A597724. The devices are suitable also for use in use in non-healthcare industries like food preparation, agriculture, and animal farming. The devices are preferably small in size (<70×40×160 mm) and perform the assays described herein in less than two hours from the moment a sample is introduced. The devices contain active elements such as pumps, valves, optics, detectors, and electronics to introduce, manipulate, and interrogate samples. The devices may provide the capability to analyze one or multiple samples simultaneously.

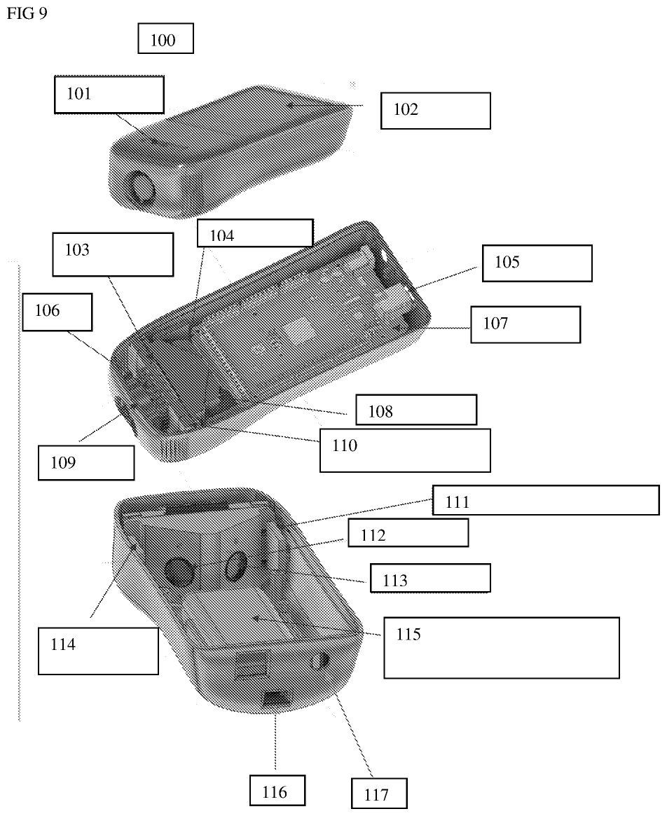

FIG. 9 is an example according to various embodiments, illustrating photographs of a handheld Real-time PCR device 100, having an external opening 101 to insert a filter paper into a PCR chamber and a touch panel 102. Inside the handheld Real-time PCR device is a PCR chamber 103 situated next to a Peltier device 110 with a temperature sensor, a heatsink 106, and a cooling fan 109. The reactions performed in the PCR chamber are measured by an optical detection system composed of a blue LED 108, an excitation filter 112, a photosensor 104, and an emission filter 113. The PCR chamber and optical detection system are regulated by a control system. The control system is comprised of a LED and photosensor control board 111 and a Peltier and cooling fan controller 114 connected to a master control board 107 connected to a communication interface 105. In a specific embodiment, the communication interface 105 is a USB port. The handheld Real-time PCR device 100 has a power system comprised of a power source 115, an external charging port 116, and a power control switch 117.

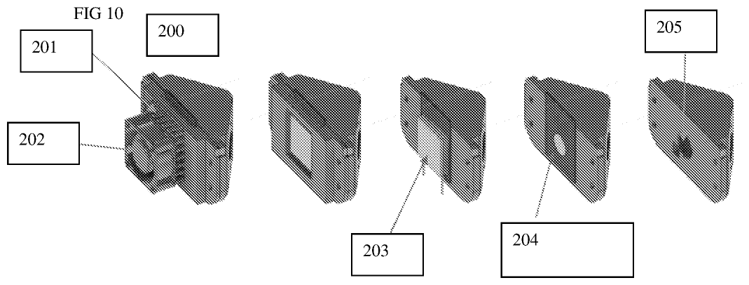

FIG. 10 is an example according to various embodiments, illustrating photographs of the PCR chamber 200. A cooling fan 202 and heatsink 201 may be used to rapidly decrease the temperature of the PCR chamber. The PCR chamber contains a Peltier device 203 which allows for rapid heating of the PCR chamber. The PCR chamber contains an external opening to allow for the insertion of a filter paper 204 containing a sample and PCR cocktail. A channel 205 into the PCR chamber gives the optical detection system access to excite and detect a fluorescence signal from a sample.

### Methods of PCR in Handheld Real-time PCR device

Further disclosed are methods for performing PCR on filter paper in the handheld Real-time PCR device. A sample is dropped to the filter paper. In some embodiments, the amount of sample is 1 μL to 20 μL. In some embodiments, the filter paper is heated to 95° C. for 5 minutes to disturb cells, bacterial and viral particles, and denature enzyme and proteins in the sample.

In some embodiments, a PCR cocktail added to the sample on the filter paper. The PCR reagents used in the present disclosure include at least a set of primers, a polymerase, various dNTPs (deoxynucleoside triphosphates), an intercalator or a fluorescently labeled probe. The cocktail also typically also contains a buffer. As a buffer, it acts to suppress the action of substances that inhibit the DNA amplification reaction, such as positively charged substances (certain proteins, etc.) and negatively charged substances (certain sugars, pigments, etc.) that exist in the body fluids of living organisms. Examples of commercially available products include Applied Biosystems™ Fast SYBR™ Green Master Mix, Invitrogen™ SuperScript™ III Platinum™ SYBR™ Green One-Step qRT-PCR Kit, or Saphir Bst2.0 Turbo GreenMaster highROX master mix.

In some embodiments, the filter paper is placed in the spacer and sealed in a plastic sheet minimizing the air before PCR is performed. The filter paper is inserted into a flat chamber in the handheld Real-time PCR device. PCR is a technique well known in the arts that consists of a series of 20-40 repeated temperature changes, called thermal cycles, with each cycle commonly consisting of two or three discrete temperature steps. The cycling is often preceded by a single temperature step at a very high temperature (>90° C. (194° F.)), and followed by one hold at the end for final product extension or brief storage. The temperatures used and the length of time they are applied in each cycle depend on a variety of parameters, including the enzyme used for DNA synthesis, the concentration of bivalent ions and dNTPs in the reaction, and the melting temperature (Tm) of the primers. The handheld Real-time PCR device contains a Peltier device that rapidly adjust the temperature, and one cycle of PCR in the device can be less than 40 secs.

In a specific embodiment, the PCR assay performed in the Real-time PCR device is isothermal PCR. Isothermal PCR is a method that can amplify nucleic acids exponentially at constant temperature, eliminating the need for thermocycler equipment. In isothermal PCR, the DNA polymerase used has high strand displacement activity, and the denature step is not required in an amplification cycle. DNA polymerases used for isothermal PCR include, but are not limited to Klenow exo-, Bsu large fragment, Bst, or Phi29. To detect RNA species, a reverse transcriptase compatible with the temperature of the reaction is added to maintain the isothermal nature of the amplification. In a specific embodiment, the isothermal PCR is performed at a temperature between about 60° C. to 70° C. In a specific embodiment, the isothermal PCR is performed at a temperature of 60° C.

### EXAMPLES

### Example 1. Standard/regular PCR

Human corona virus 229E (HCoV 229E: ATCC® VR-740™) and human lung fibroblast cell line MRC-5 (ATCC® CCL-171™) are used in the experiments. For expansion, MRC-5 cells are cultured in a cell treated T-75 flask with DMEM supplemented with 10% FBS, and 1% Penicillin-Streptomycin. Cells are incubated at 37° C. with 5% CO2. MRC-5 cells are trypsinized and moved to a tissue culture-treated 6-well cell culture plate. The cells are then inoculated with HCoV-229E virus in a media containing DMEM supplemented with 2% FBS. The incubation temperature is lowered to 35° C. for 2-5 days. Once signs of cytopathic effect (CPE) are observed, virus containing media is collected in conical tubes and stored at 4° C. (−80° C. for longer storage).

Total RNA samples as well as cDNA (complimentary DNA synthesized using mRNA template) are purified from human neural stem cells (NSC, procured as Fetal-derived human neural progenitor cells from Lonza) and Glioblastoma multiforme (GBM) cells. Primary GBM cell culture is prepared by dissociation of human brain tumor patient specimens in accordance with a protocol approved by Florida Hospital Institutional Review Board. The subjects are given informed consent and HIPPA regulations is strictly followed. For proliferation, the cells are cultured in HNSC media containing Heparin 5000 U (0.5 U/mL), EGF—20 ng/ml, bFGF—20 ng/ml and 2% B27 stock mixed in DMEM/F12. To differentiate these cells, the cells are cultured in NT2 (NTERA-2 human embryonal carcinoma cell line) media containing DMEM-F12 supplemented with 10% exosome-depleted FBS.

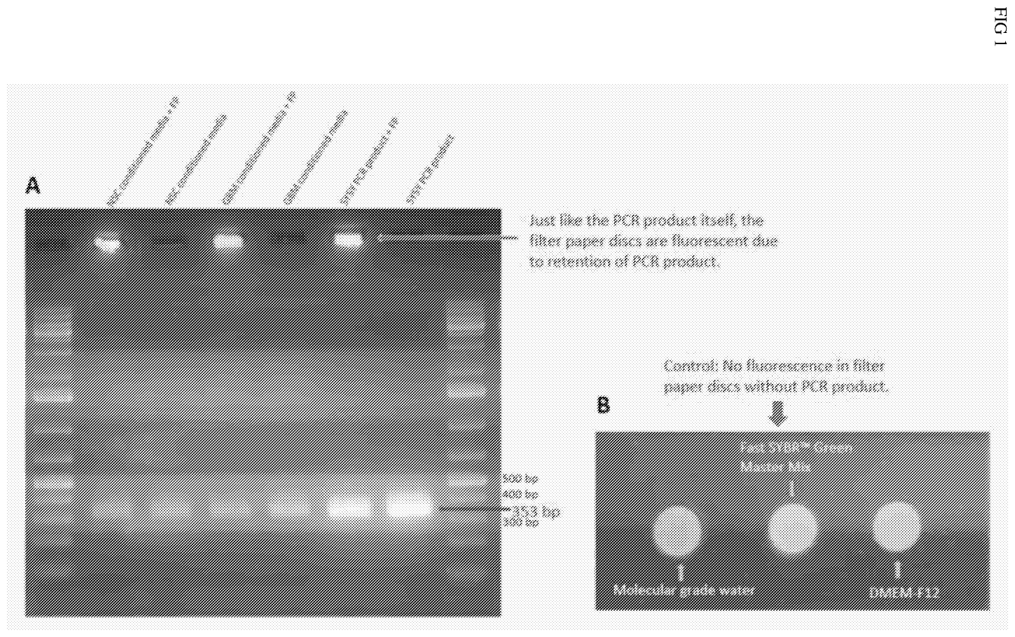

For this PCR amplification and the rest, Fisher-brand quantitative Q5 filter paper (FP) with medium porosity (Fisher #09-790-2G) is used for the trial. Six mm circular discs are punched out to have the uniformity of size. Instead of isolating the DNA, or removing the cell debris and macromolecules by centrifugation, 10 μl of conditioned media from NSC and GBM cell cultures, which contain exosomal DNA, is pipetted directly on the paper disc, followed by heating it at 95° C. for 5 min. The PCR reactions are carried out in the PCR-microtube with the amplification reaction mixture. The reaction mixture comprised of MgCl2, dNTPs, humanSOX2 primers (Forward: 5′-CCCCCTTTATTTTCCGTAGTT-3′/Reverse: 5′-5′ ATCATCCAGCCGTTTCTTTTT-3′), GoTaq® G2 Flexi DNA Polymerase (Promega #M7801) and 5X flexi buffer. As a positive control, a PCR product of human neuroblastoma cell line SY5Y cytoplasmic DNA previously amplified with the same SOX2 primers is used. Each template is amplified on filter paper disc, as well as without the disc, as control. The samples are subjected to thermo-cycling at 94° C.—5 minutes, (denaturation: 94° C.—30 seconds, annealing: 48° C.—30 seconds, Extension: 72° C.—2 minutes)x30, 72° C.—10 minutes. The PCR products are mixed with 6X orange loading dye and electrophoresed in SYBR-safe containing. 1.5% Agarose gel in 1X TAE buffer. The PCR products are visualized under UV light and the fluorescence of DNA binding dye SYBR™ Safe DNA Gel Stain (Thermo Fisher #S33102) is imaged at an excitation/emission wavelength 470/530 nm (FIG. 1A).

The nucleic acids are amplified from 10 μL sample taken from a culture flask containing approximately 107 cells in 20 ml of conditioned media. Even though the sample volume of 10 μl contains miniscule amounts of DNA, with “PCR on paper” technique, the DNA does not need to be purified or concentrated from large volumes of conditioned media and can be sufficiently amplified making the PCR product detectable. Though the PCR bands from the samples amplified without paper show equal signal strength as that of the bands of PCR product amplified on filter paper, it is important to note that the filter paper itself holds DNA to show fluorescence signals. This is a clear proof that many folds more amplification of DNA can be achieved on paper than without it, under similar experimental conditions and equal amount of template DNA (FIG. 1A). The source of cell-free DNA in conditioned media is possibly nano-vesicles such as exosomes, extracellular microvesicles (30-100 nm) released by almost all types of cells upon fusion of its multi-vesicular body with the plasma membrane. In the published data. it has already been shown that the exosomal DNA can be amplified without isolation [Vaidya M. Bacchus M. Sugaya K. Differential sequences of exosomal NANOG DNA as a potential diagnostic cancer marker. PloS one. 2018 May 22; 13(5):e0197782.].

### Example 2. Real time PCR

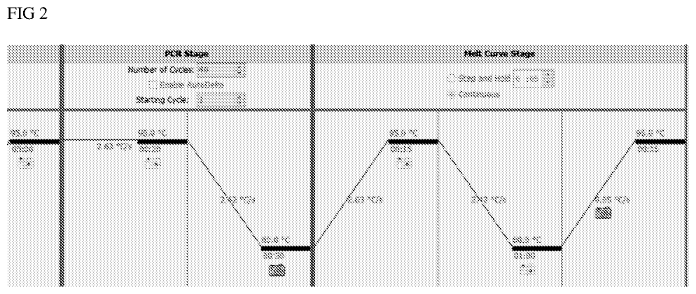

For real time PCR amplification, instead of isolating the DNA, or removing the cell debris and macromolecules by centrifugation, 5 μl of conditioned media from NSC and GBM cell cultures is pipetted directly on the paper disc, followed by heating it at 95° C. for 5 min. The reactions are carried out in the qPCR-specific microplate after adding the PCR reagent to the dried filter paper. The reaction mixture comprised of Applied Biosystems™ Fast SYBR™ Green Master Mix (cat #43-856-12) and q-PCR-specific primers for (human B-ACTIN primers (Forward: 5′-AGAGCTACGAGCTGCCTGAC-3′/Reverse: 5′-AGCACTGTGTTGGCGTACAG-3′). Each template is amplified on filter paper disc. As a control, 5 μl of conditioned media from NSC and GBM cell cultures is added to PCR reaction in a tube. The samples are subjected to thermo-cycling using Applied Biosystems QuanStudio 7 Flex. The thermocycling run method is described in FIG. 2. Real-time PCR device displaying the run method and data from a SYBR green real time PCR.

A significant difference of CT mean values between the experimental samples (NSC and GBM media) and the controls (DMEM-F12) indicates that the DNA molecules from the conditioned media were successfully amplified without their purification or isolation. DNA present in as low as 5 μl media yielded a successful amplification. As explained in carlier section, a successful amplification of minute amounts of DNA present in 5 μl sample taken from a culture flask containing approximately 107 cells in 20 ml of conditioned media shows the high efficacy of the amplification procedure. Even though the sample volume of 5 μl contains miniscule amounts of DNA, measurable amplification of the template using paper, where the DNA does not need to be purified or concentrated from large volumes of conditioned media, is a successful of proof of concept. With regular PCR involving isolation of nucleotide will not be able to amplify from such a small sample containing very low amount of nucleotides because a loss of nucleotide during the isolation process. Though the CT mean values of GBM media and NSC media are close to their respective CT values for amplification on filter paper, it is important to note that if the DNA in 5 μl volume happens to be insufficient to have recordable amplification, the filter paper can accommodate more volume by drying the previously applied sample. This potentially increases the template DNA amount making the filter paper more useful for quantitative as well as qualitative amplification without DNA isolation. In contrast, due to the limit on total volume per PCR reaction in a thermocycler, the volume of conditioned media cannot exceed a certain amount.

### Example 3. Reverse Transcriptase PCR

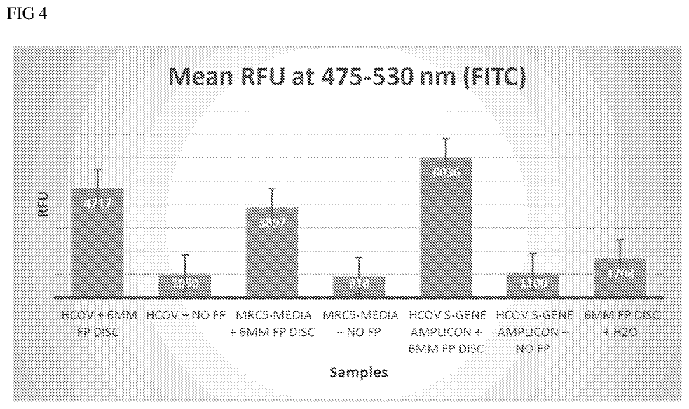

Reverse transcription of RNA and its subsequent amplification is achieved using Invitrogen™ M SuperScript™ III Platinum™ SYBRTM Green One-Step qRT-PCR Kit (Fisher Scientific #11-736-051). On 6 mm filter paper discs, 8 μl of HCoV229E containing conditioned media of human lung-fibroblast cells (MRC-5) is directly applied to the filter paper. The subsequent heating processes (95° C. for 5 minutes), which also deactivate viral particles and thermocycling for amplification are carried out on the same filter paper, in a PCR tube. The reaction mix comprised of SuperScript™ III Platinum™ RT Taq mix, 2X SYBR™ Green reaction mix, random hexamers, ROX as reference dye and molecular grade water. The transcribed template cDNA is amplified with HCoV229E S-gene primers (Forward: 5′-CGTTGACTTCAAACCTCAGA-3′/Reverse: 5′-ACCAACATTGGCATAAACAG-3′). In a standard thermocycler, the samples are subjected to thermo-cycling at 50° C. for 5 minutes for cDNA synthesis, initial denaturation at 95° C. for 5 minutes, (denaturation: 95° C.—15 seconds, annealing: 60° C.—30 seconds, Extension: 72° C.—45 seconds) x 40, and a final extension at 72° C. for 10 minutes. As controls, plain MRC media and purified PCR product of HCoV229E-S gene are used as templates. Upon thermocycling, the samples are transferred to a flat-bottom 96-well plate and the fluorescence is measured from top at FITC excitation/emission of 475/530 nm in a microplate reader. The mean relative fluorescence unit (RFU) values are compared with control samples. The significantly higher RFU values of HcoV229E samples as compared to the controls indicates a successful conversion of viral RNA followed by amplification of the S-gene, without having to isolate and purify the viral particles (Table 1, FIG. 4).

TABLE 1
RFU values of the samples at excitation and emission wavelengths
of 475/530 nm. (RFU—relative fluorescence units, HcoV—Human
CoV- 229E, FP—filter paper). After amplification reactions
was carried out in a standard thermocycler, the samples were
transferred to a 96-well plate to record fluorescence.

| Sample | Mean RFU [Read 475, 530] |
|---|---|
| HcoV + 6 mm FP disc | 4717 |
| HcoV -without using FP | 1050 |
| MRC5-media + 6 mm FP disc | 3897 |
| MRC5-media -without using FP | 918 |
| HcoV S-gene amplicon + 6 mm | 6036 |
| FP disc | |
| HcoV S-gene amplicon - | 1100 |
| without using FP | |
| 6 mm FP disc + H₂O | 1708 |

Eight μl of HCoV229E RNA viral particles in lung fibroblast media can be detected without isolation of nucleotides by one-step PCR amplification. Comparison of RFU values showed that no more than fluorescent signal (no amplification of DNA) without the flier paper. It indicates that the filter paper aids to eliminate the substances inhibiting PCR reaction and successful amplification of the target RNA in one-step PCR. This technology would be very useful for diagnosis of COVID eliminating the isolation process of nucleotide, which needs well equipped laboratory and skilled technicians.

### Example 4. Isothermal PCR

As the name indicates, the isothermal PCR uses a steady single temperature not exceeding 70° C. (60° C. for the experiment) and a special, heat resistant DNA Bst polymerase with stand displacement activity. The new strand is synthesized while the enzyme dissociates hydrogen bonds of double stranded DNA template. Quantitative detection of the amplification product can be achieved with DNA-intercalating fluorescent dye like SYBR green. To kickstart the displacement, the polymerase requires “bump” primers and the rest of the amplification uses “extension” primers for synthesizing a full PCR product. Since Bst polymerase exhibits high tolerance to inhibitors typically present in diagnostic specimens, its use is desirable for DNA amplification in various clinical purposes (https://www.neb-online.de/wp-content/uploads/2015/04/NEB_isothermal_amp.pdf).

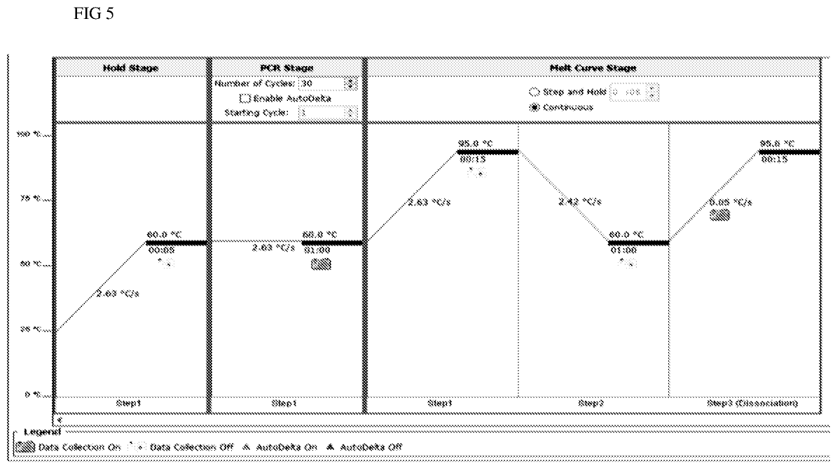

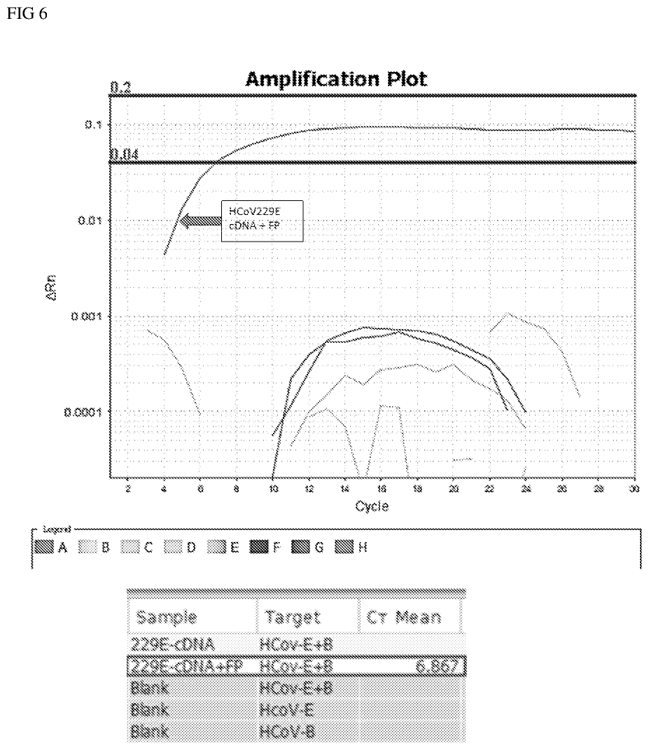

To get rid of the constraints of thermocycling where the filter paper periodically gets exposed to high temperatures of 90° C. for denaturation of the double strands of cDNA, isothermal PCR is conducted on the cDNA samples using Saphir Bst2.0 Turbo GreenMaster highROX master mix (Jenna Bioscience #PCR394S). Eleven μl of HCoV229E virus particle containing human lung fibroblast MRC-5 media is added to the filter paper and subsequent heat-inactivation (95° C. for 5 min), cDNA, isothermal PCR and standard PCR (on isothermal PCR product) are carried out on the same filter paper without isolation of the virus from the media. The PCR mix contains Bst polymerase with its buffer, dNTPs, highROX and two sets of primers (bump primers (B) F: 5′-GTGTTGTACGTTGACTTCAAACCTCAGA-3′, R: 5′-CTCCACCTACCAACATTGGCATAAACAG-3′ and extension primers (E) F: 5′-GTGTTGTAGAGTCACTCTCGTTGAC 3′, R: R: 5′-CTCCACCTCTGAGAAAATACCAACA 3′) specific for S-gene. The mix also contains the fluorescent DNA stain EvaGreen® that intercalates into DNA during the amplification process and allows the direct quantification of target DNA by fluorescence detection analogous to real-time PCR. (Jenna Bioscience website). cDNA is synthesized using total RNA of the virus HCoV229E and GBM/NSC cellular RNA using Invitrogen™ SuperScript™ IV Reverse Transcriptase (Invitrogen #18-090-010). Virus particles are inactivated at 95° C. for 5 minutes (while on filter paper). cDNA synthesis is carried out on filter paper by directly adding the reaction mix to the paper. Isothermal PCR is carried out on the filter paper at 600C (FIG. 5). The CT mean values indicate a successful cDNA synthesis and amplification. Details are in FIG. 6.

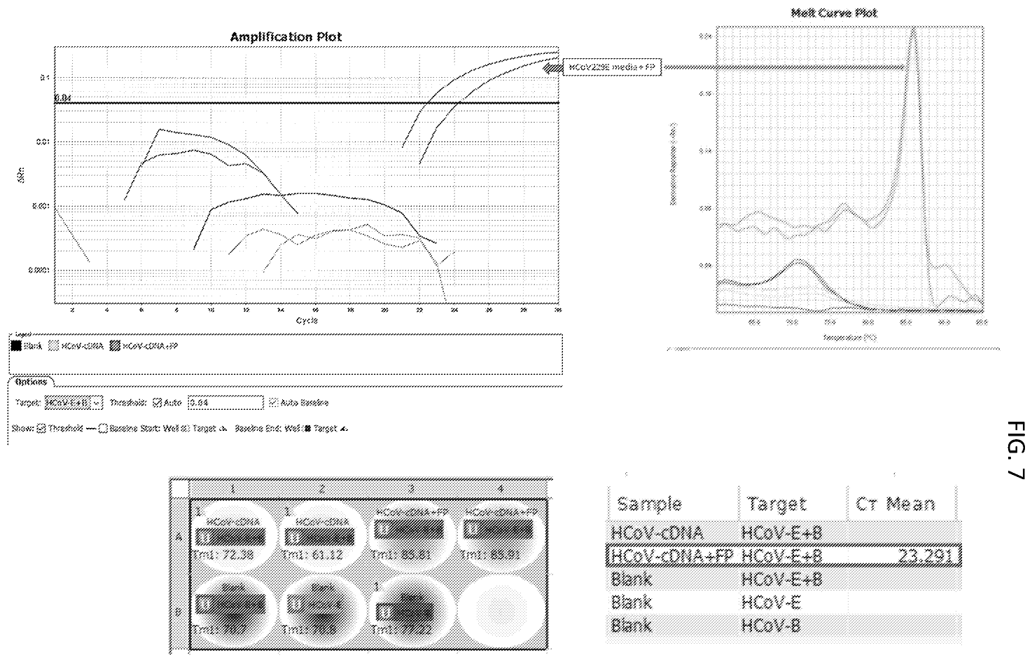

In a repeated experiment heat inactivation of viral particles and the subsequent cDNA synthesis and Isothermal PCR is carried out on filter paper. S-gene is amplified again using Bump primers (B) along with the extension primers (E)—at a double concentration than the previous experiment. Considering the CT mean value, the amplification of viral S gene was successful (FIG. 7).

Just as in reverse transcriptase PCR, viral RNA from small amount of conditioned media, which contains minimal amount of viral particles, could be converted to cDNA and successfully amplified at single temperature of 60° C. The CT mean values of the reactions performed on filter paper compared to the mean values of the reactions performed without filter paper indicate that the filter paper is an essential part of the process for successful amplification of the viral cDNA. All the processes such as sample collection, viral inactivation and the amplification performed on the same piece of paper is a proof of concept that is put forth in this disclosure.

### Example 5: Filter with immobilized capture molecules

Further to the Examples above that enable PCR without isolation of nucleotides using filter as the reaction chamber, this Example presents a modified filter to capture pathogen more efficiently from the large volume of media such as air and liquid. For example, the typical paper used for PCR reaction chamber size is rather small and it may only hold up to 50 μl of the volume. Accordingly, when there is a smaller water sample (e.g., 100 ml) which may be contaminated with a limited number of viral particles or other pathogen (e.g. 1000 viral particles) then the chance of microliter sample (e.g. 50 μl of the sample) to contain a virus particle is very low (50/50). However, if the solution is filtered with filter modified with aptamers or antibodies to capture the target virus, and in turn, PCT is conducted with this filter paper, then the chances of detecting virus in the samples increases dramatically.

Another benefit of using modified filters to capture pathogen is that it enables quantification of live virus versus total target nucleotide sequence. To illustrate using the example of SARS-COV-2, aptamers or antibody recognizing viral shell or spike proteins of this virus are immobilized on a filter. This filter will capture only the shell of the virus. When PCR is conducted on this preparation, this will enable detection of live virus having both shell and genome, and dead viral genome in the sample can be excluded. This allows quantification of a pathogenic threat rather than just total viral genome nucleic acids, which may not cause any problem.

In one specific embodiment, pathogen is captured on cellulose filter paper using biosensing molecules, such as aptamers and antibodies. These molecules may be immobilized on the filter in such a fashion so that they can bind to the target. There are many conventional methods for attaching antibodies or aptamers to cellulose filter paper. In an even more specific embodiment, antibodies or aptamers recognizing target pathogen surface protein (e.g. spike protein of SARS-COV-2) are immobilized on filter paper (e.g. cellulose filter paper).

One example of immobilizing antibodies onto cellulose filter paper is set forth in the following the published article: Peng Y, Gelder VV, Amaladoss A, Patel KH. Covalent Binding of Antibodies to Cellulose Paper Dises and Their Applications in Naked-eye Colorimetric Immunoassays. J Vis Exp. 2016; (116):54111. Published 2016 Oct. 21. doi:10.3791/54111. The summary of the immobilization technique is provided in the following diagram:

An example of immobilization of aptamer onto cellulose filter paper is provided in the following publication: Su S., Nutiu R., Filipe C.D.M., Li Y., Pelton R. Langmuir. 2007;23:1300. Briefly, immobilization is achieved by oxidization of regenerated cellulose to give aldehyde groups. These aldehyde groups are reacted with amine groups on a DNA aptamer to form a Schiff base, which is then reduced to give a stable covalent bond. The following diagram of the Su et al. paper summarizes this technique:

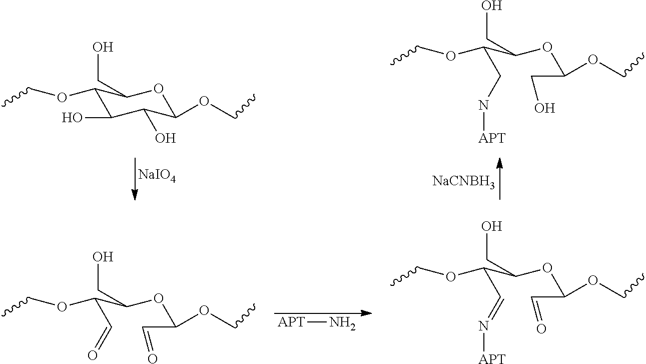

In one specific example, the modified filters are used to filter a sample solution. Alternatively, to capture pathogen from air, the filter may be wetted with a buffer, such as PBS. After the filtrations, the filter is dried up and heated at 95° C. for at least 5 min to deactivate the pathogen, which also exposes the nucleic acids contained by the pathogen. This procedure enables PCR to be conducted directly on the filter as described in the Examples described above.

In other embodiments, the biosensing molecules (capture molecules) are conjugated with a large molecule, particles or beads and mixed with the sample under conditions to bind to a target pathogen. In this situation, regular filter paper may be used to filter out the conjugated biosensing molecules bound with the target pathogen. After the filtrations, similar to that described above, the filter is dried up and heated at 95° C. for at least 5 min to deactivate the pathogen, which exposes the nucleic acids contained by the pathogen. PCR directly may be conducted on the filter as described above.

Current PCR technology requires extraction and purification of target nucleotides. This process requires a specialized facilities, skilled technicians, and expensive equipment. Also, current PCR techniques tend to lose significant amount of target nucleotides, which reduces the sensitivity. The embodiments disclosed herein will eliminate those issues associated with the current PCR technology and significantly improve sensitivity by direct sampling and amplification on the same filter papers.

According to one embodiment, provided is a method for amplifying and detecting one or more nucleic acid targets in a sample. The method involves collecting a sample on a filter paper comprising one or more capture molecules immobilized thereon, heat inactivating the sample on the filter paper, applying a PCR cocktail to the sample on the filter paper, inserting the filter paper into a PCR device, performing one or more amplification cycles using the PCR device, and detecting the presence or absence of one or more nucleic acid target.

Another embodiment pertains to a cellulose filter paper comprising an antibody and/or aptamer immobilized onto a surface thereof. The antibody and/or aptamer may be specific to a viral surface protein. In a specific embodiment, the viral surface protein is SARS-COV-2 spike protein. The modified cellulose filter paper may be configured for insertion into a PCR device such as a handheld, Real-time PCR device.

What is claimed is:

1. A method for amplifying and detecting one or more nucleic acid targets in a sample comprising: collecting a sample, applying the sample to a filter paper, heat inactivating the sample on the filter paper, applying a PCR cocktail to the sample on the filter paper, inserting the filter paper into a handheld Real-time PCR device, performing one or more amplification cycles using the handheld Real-time PCR device, and detecting the presence or absence of one or more nucleic acid target.

2. The method of claim 1, wherein the sample comprises blood, serum, saliva, sputum, mucus, urine, milk, or any liquid containing nucleic acids.

3. The method of claim 1, wherein the filter paper comprises quantitative filter paper.

4. The method of claim 1, wherein the heat inactivating comprises incubating the sample on the filter paper at 95° C. for at least 5 minutes.

5. The method of claim 1, wherein the PCR cocktail comprises at least one primer pair for priming amplification of a nucleic acid target, an enzyme for amplifying the nucleic acid targets, dNTPs, and a reaction buffer.

6. A method for amplifying and quantifying one or more nucleic acid targets in a sample comprising collecting a sample, applying the sample to a filter paper, heat inactivating the sample, applying a PCR cocktail and a labeling agent to the sample on the filter paper, inserting the filter paper into a handheld Real-time PCR device, performing one or more amplification cycles using the handheld Real-time PCR device to form labeled amplification products for each of the one or more nucleic acid targets, and detecting and quantifying a signal from the labeled amplification products.

7. The method of claim 5, wherein the sample comprises blood, serum, saliva, sputum, mucus, urine, milk, or any liquid containing nucleic acids, or any sample collected from a surface, such as swipe sampling.

8. The method of claim 5, wherein the filter paper comprises quantitative filter paper.

9. The method of claim 5, wherein the heat inactivating comprises incubating the sample on the filter paper at 95° C. for at least 5 minutes.

10. The method of claim 5, wherein the PCR cocktail comprises at least one primer pair for priming amplification of a nucleic acid target, an enzyme for amplifying the nucleic acid targets, nucleotides, and a reaction buffer.

11. The method of claim 10, wherein the enzyme for amplifying the nucleic acid targets comprises a heat resistant and strand-displacement DNA polymerase.

12. The method of claim 5, wherein the labeling agent comprises a fluorescent label selected from the group comprising SYBR™ Green or EvaGreen®.

13. The method of claim 5, wherein the amplification cycles do not exceed a temperature of 70° C.

14. A handheld Real-time PCR device, comprising:
a. a PCR chamber to hold a filter containing a sample and a PCR cocktail:
comprising a peltier device connected to a heatsink and a cooling fan;
b. an optical detection system comprising a blue LED, a photosensor, an excitation filter, and an emission filter;
c. a control system comprising a Peltier and cooling fan controller, a LED and photosensor control board, a master control board connecting to an exterior touch control panel, and a communication interface to interact with other devices; and
d. a power system comprising a power source, an external charging port, and a power control switch.

15. The device of claim 14, wherein the PCR chamber has an opening allowing for inserting the filter containing a sample and a PCR cocktail.

16. The device of claim 14, wherein the heatsink and cooling fan direct heat from the PCR chamber to outside the device.

17. The device of claim 14, wherein the optical detection system excites and detects a fluorescence signal from a sample within the PCR chamber.

18. The device of claim 14, wherein the excitation filter comprises a band pass 480 nm filter.

19. The device of claim 14, wherein the emission filter comprises a band pass 520 nm filter.

20. The device of claim 14, wherein the control system is used for programing amplification cycles for the PCR chamber.

21. The device of claim 14, wherein the exterior touch control panel displays PCR settings and data, or wherein the exterior touch control panel is associated with a separate device that communicates with the master control board, wherein the separate device is optionally a smartphone.

22. The device of claim 14, wherein the power source comprises a rechargeable battery or non-rechargeable battery.

23. The device of claim 14, wherein the battery last for at least 2, at least 3, at least 4, at least 5 or at least 6 PCR reactions.

24. The device of claim 12, wherein the communication interface is selected from the group comprising serial, USB, Bluetooth, wifi, cellular, satellite, or combinations thereof.

25. The method of any of claims 1-13, wherein amplicons are detected by fluorescence.

26. The method of claim 25 where in fluorescence is produced from eitherDNA binding dye, such as SYBR-green and fluorescence dye conjugated probe used in Taq-man PCR.

27. A method for amplifying and detecting one or more nucleic acid targets in a sample comprising: collecting a sample on a filter paper comprising one or more capture molecules immobilized thereon, heat inactivating the sample on the filter paper, applying a PCR cocktail to the sample on the filter paper, inserting the filter paper into a Real-time PCR device, performing one or more amplification cycles using the Real-time PCR device, and detecting the presence or absence of one or more nucleic acid target.

28. The method of claim 27, wherein the capture molecule is an antibody and/or aptamer, that optionally binds to an antigen such as a nucleic acid or amino acid sequence.

29. The method of claim 28, wherein the antigen is of a microbe such as virus, fungi or bacterium, wherein optionally, the microbe is a pathogen.

30. The method of claim 29, wherein the pathogen is a virus comprising a surface protein.

31. The method of claim 30, wherein the antibody and/or aptamer binds to the surface protein.

32. The method of any of claim 30 or 31, wherein the surface protein is spike protein of SARS-COV-2.

33. A method for amplifying and quantifying one or more nucleic acid targets in a sample comprising collecting a sample on a filter paper comprising one or more capture molecules immobilized on a surface thereof, heat inactivating the sample, applying a PCR cocktail and a labeling agent to the sample on the filter paper, inserting the filter paper into a Real-time PCR device, performing one or more amplification cycles using the Real-time PCR device to form labeled amplification products for each of the one or more nucleic acid targets, and detecting and quantifying a signal from the labeled amplification products.

34. The method of claim 33, wherein the capture molecule is an antibody and/or aptamer.

35. The method of claim 34, wherein the antibody and/or aptamer binds to a pathogen.

36. The method of claim 35, wherein the pathogen is a virus comprising a surface protein.

37. The method of claim 36, wherein the antibody and/or aptamer binds to the surface protein.

38. The method of any of claim 35 or 36, wherein the surface protein is spike protein of SARS-COV-2.

39. A cellulose filter paper comprising an antibody and/or aptamer immobilized onto a surface thereof.

40. The cellulose filter paper of claim 39, wherein the antibody and/or aptamer is specific to a viral surface protein.

41. The cellulose filter paper of claim 40, wherein the viral surface protein is SARS-CoV-2 spike protein.

42. The cellulose filter paper of any of claims 39-41 disposed in a device according to any of claims 14-24.

43. The cellulose filter paper of any of claims 39-41 contained in a package bearing a label indicating its contents.

44. The method of any of claim 1-13 or 27-38, wherein the filter paper is white or a color other than white.

45. The method of claim 44, wherein the filter paper is black, brown, blue, green or grey or a combination thereof.

46. The cellulose filter paper of any of claims 39-43, wherein the filter paper is white or a color other than white.

47. The cellulose filter paper of claim 46, wherein the filter paper is of a color to enhance detection of a fluorescence.

48. The cellulose paper of claim 46, wherein the filter paper is black, brown, blue, green or grey or a combination thereof.
